(19) United States
(12) Patent Application Publication
(10) Pub. No.: US 2024/0342217 A1
(43) Pub. Date: Oct. 17, 2024

(54) ANTI-INFLAMMATORY COMPOSITION COMPRISING EXTRACELLULAR VESICLES ISOLATED FROM TONSIL-DERIVED MESENCHYMAL STEM CELLS AS AN ACTIVE INGREDIENT

(71) Applicant: HYUNDAEMEDITECH CO., LTD., Wonju-si (KR)

(72) Inventor: Mi Hee SONG, Wonju-si (KR)

(21) Appl. No.: 18/252,871

(22) PCT Filed: Oct. 26, 2022
(86) PCT No.: PCT/KR2022/016439

§ 371 Date: May 12, 2023

(30) Foreign Application Priority Data
Sep. 7, 2022 (KR) 10-2022-0113438

(51) Int. Cl. A61K 35/28; A61K 8/98; A61P 29/00; A61Q 19/00

(52) U.S. Cl. CPC A61K 35/28; A61K 8/981; A61P 29/00; A61Q 19/00

(57) ABSTRACT

The present invention relates to an anti-inflammatory active composition comprising extracellular vesicles isolated from tonsil-derived mesenchymal stem cells. Specifically, the anti-inflammatory active composition comprising extracellular vesicles isolated from tonsil-derived mesenchymal stem cells of the present invention has an excellent activity of inhibiting TNF-α, which is an inflammatory cytokine, and an excellent activity of increasing IL-10, which is an anti-inflammatory cytokine, and thus it may be usefully applied to the prevention, suppression, alleviation, amelioration or treatment of inflammation.

### TECHNICAL FIELD

The present invention relates to an anti-inflammatory composition comprising extracellular vesicles isolated from tonsil-derived mesenchymal stem cells as an active ingredient.

### BACKGROUND ART

Extracellular vesicles are nano-sized vesicles that are secreted by all cells into the external environment for intercellular information exchange. Extracellular vesicles contain various substances exhibiting biological activity, such as proteins, lipids, nucleic acids, and metabolites. Recently, substances constituting extracellular vesicles have been identified through high-throughput analysis. In addition, extracellular vesicles reflect the conditions of the cells from which they are derived and have been found in various body fluids. While interacting with target cells, extracellular vesicles perform physiological functions under normal conditions and pathological functions that cause diseases. Recently, studies on the diagnosis and treatment of diseases using extracellular vesicles have been actively conducted.

On the other hand, inflammation is a defensive response of a living body that appears in response to pathological conditions caused by physical or chemical trauma, infection by bacteria, fungi, or viruses, various allergens, and the like. The inflammatory response occurs as part of the innate immune response. Various substances and physiological and chemical phenomena are involved in the inflammatory response, and recent studies have showed that various inflammatory cytokines play an important role in the inflammatory response. Major cytokines that are involved in the inflammatory response include IL-10, TNF-α, IL-6, IL-8, IL-12, and IFN-0, and an increase of their expression and secretion as well as their activation is associated with a series of complex physiological reactions, such as secretion of inflammation-mediating substances, immune cell infiltration, cell migration, and tissue destruction, and symptoms including erythema, edema, fever, and pain.

In general, when the source of infection is removed and the damaged tissue is regenerated, an inflammatory response does not become a major problem and the diseased area is restored to the normal conditions. However, when the source of infection is not removed or an inflammation occurs excessively or continuously by an internal substance, acute or chronic inflammatory is caused.

For the alleviation or treatment of inflammatory responses or inflammatory diseases caused thereby, immunosuppressants such as nonsteroidal anti-inflammatory drugs, steroidal anti-inflammatory drugs, neuropeptide antagonists, COX inhibitors, antihistamines, and cyclosporin A are used, they cause side effects such as atrophoderma, vasodilation, depigmentation, hypersensitivity, tolerance, and neutropenia.

Therefore, there is a need to overcome the problems of existing therapeutic agents and to develop substances having excellent anti-inflammatory effects.

### DETAILED DESCRIPTION OF THE INVENTION

### Technical Problem

The purpose of the present invention is to provide an anti-inflammatory active composition comprising extracellular vesicles isolated from tonsil-derived mesenchymal stem cells to solve the problems described above.

Another purpose of the present invention is to provide a pharmaceutical composition for preventing or treating inflammatory diseases, the pharmaceutical composition comprising the anti-inflammatory active composition.

Another purpose of the present invention is to provide an anti-inflammatory functional cosmetic composition comprising the anti-inflammatory active composition.

Another purpose of the present invention is to provide a health functional food composition for preventing or ameliorating inflammatory diseases, the health functional food composition comprising the anti-inflammatory active composition.

Another purpose of the present invention is to provide a quasi-drug composition for preventing or ameliorating inflammatory diseases, the quasi-drug composition comprising the anti-inflammatory active composition.

### Technical Solution

To solve the problems described above, the present invention provides an anti-inflammatory active composition comprising extracellular vesicles isolated from tonsil-derived mesenchymal stem cells to solve the problems described above.

In addition, the present invention provides a pharmaceutical composition for preventing or treating inflammatory diseases, the pharmaceutical composition comprising the anti-inflammatory active composition.

In addition, the present invention provides an anti-inflammatory functional cosmetic composition comprising the anti-inflammatory active composition.

In addition, the present invention provides a health functional food composition for preventing or ameliorating inflammatory diseases, the health functional food composition comprising the anti-inflammatory active composition.

In addition, the present invention provides a quasi-drug composition for preventing or ameliorating inflammatory diseases, the quasi-drug composition comprising the anti-inflammatory active composition.

Hereinafter, the present invention will be described in more detail through Examples. However, since these Examples are only for illustrative purposes, the scope of the present invention should not be construed as being limited by these Examples.

### Advantageous Effects

The anti-inflammatory active composition comprising extracellular vesicles isolated from tonsil-derived mesenchymal stem cells of the present invention has an excellent anti-inflammatory effect.

Therefore, an anti-inflammatory composition comprising the extracellular vesicles isolated from tonsil-derived mesenchymal stem cells as an active ingredient of the present invention can be used to effectively prevent, suppress, alleviate, ameliorate or treat inflammatory responses or inflammatory diseases.

On the other hand, the scope of the present invention is not limited by the effects as described above. The effects according to the present invention are not limited by the contents illustrated above, and more various effects are included in the present specification.

### DESCRIPTION OF THE DRAWINGS

FIGS. 1 to 4 are diagrams showing the results of the analysis of physical properties of stem cell-derived extracellular vesicles according to an Example of the present invention, presenting the size distribution and the number of the particles obtained by nanoparticle tracking analysis (NTA).

FIG. 5 is a diagram confirming and showing the TNF-α production inhibitory effect of an anti-inflammatory active composition comprising extracellular vesicles isolated from tonsil-derived mesenchymal stem cells according to an Example of the present invention.

FIG. 6 is a diagram confirming and showing whether the residual amount of IL-10 in a culture medium itself affects the results according to an Example of the present invention.

FIG. 7 is a diagram confirming and showing an IL-10 production increasing effect of an anti-inflammatory active composition comprising extracellular vesicles isolated from tonsil-derived mesenchymal stem cells according to an Example of the present invention.

### MODE OF THE INVENTION

Advantages and features of the present invention and methods for achieving the same will become clear with reference to the Examples described in details below with the accompanying drawings. However, the present invention is not limited to the Examples disclosed below, but will be implemented in a variety of different forms. These Examples make the disclosure of the present invention complete and are provided to completely inform the scope of the invention to one of ordinary skill in the art. The present invention is defined only by the scope of the claims.

The present invention is described in more detail in the Examples described below. However, the Examples described below are merely illustrative of the contents of the present invention, and are not intended to restrict or limit the scope of the present invention. What can be easily inferred by one of ordinary skill in the art from the detailed description and Examples of the present invention is interpreted as belonging to the scope of the present invention. References cited in the present invention are incorporated in the present invention as references.

Throughout the specification, when a certain component is said to “comprise”, it means that it may further comprise other components without excluding other components unless otherwise stated particularly.

As used in the present specification, the term “extracellular vesicles (EV)” comprehensively refers to cell membrane-derived membrane vesicles, ectosomes, shedding vesicles, microparticles, microvesicles, exosomes, cell secretome, or an equivalent thereof.

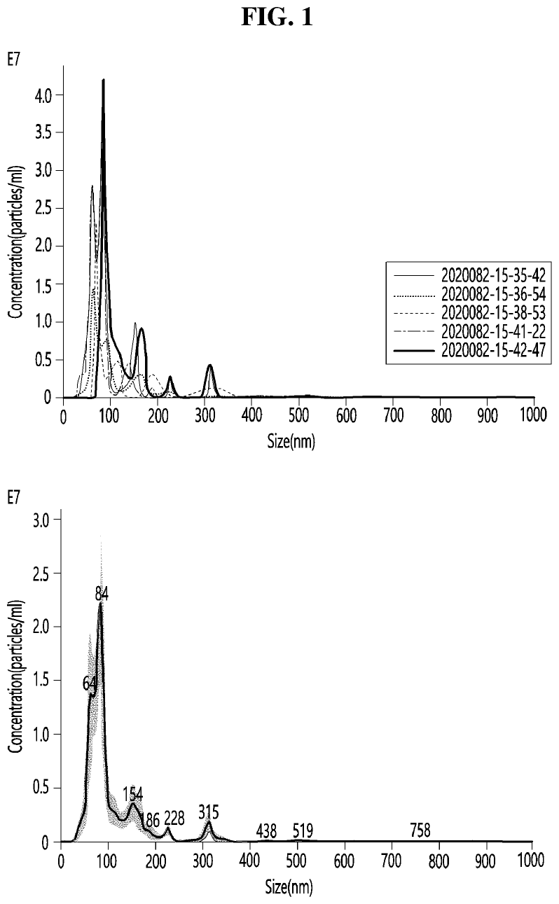

As shown in Example 3 and FIGS. 1 to 4, the particle size of the extracellular vesicle fraction of the present invention may be an average diameter of 10 to 400 nm, more preferably 50 to 300 nm, but is not limited thereto.

As used in the present specification, the term “exosomes” refers to extracellular vesicles having a size of tens to hundreds of nanometers (preferably approximately 30 to 200 nm) consisting of a phospholipid bilayer that is identical to the structure of a cell membrane (When particle size of exosomes may vary depending on the type of stem cell to be isolated, the isolation method, and the measurement method.) (Vasiliy S. Chernyshev et al., “Size and shape characterization of hydrated and desiccated exosomes”, Anal Bioanal Chem, (2015) DOI 10.1007/s00216-015-8535-3), and proteins called exosome cargo, mRNA, miRNA, DNA, etc. are included. Exosome cargos include a wide range of signaling factors, and these signaling factors are known to be cell type-specific and be differently regulated depending on the environment of the secreting cells. Exosomes are intercellular signaling mediators secreted by cells, and various cell signals transmitted through exosomes are known to regulate cell behavior, including activation, growth, migration, differentiation, dedifferentiation, apoptosis, and necrosis of target cells. The exosomes used in the present invention can be isolated from the stem cell culture medium by using various methods known in the art, but are not limited thereto. Exosomes isolated by various isolation methods described above can obviously be used in the anti-inflammatory composition of the present invention.

As used in the specification, the term “inflammation” refers to a pathological condition of an abscess formed by invasion of external sources of infection (bacteria, fungi, viruses, various types of allergens).

In addition, the present invention provides a pharmaceutical composition for preventing or treating inflammatory diseases, the pharmaceutical composition comprising the anti-inflammatory active composition described above.

As used in the present specification, the term “anti-inflammatory activity” refers to suppression of inflammation, and the inflammation, which is one of the defensive responses of living tissues to certain stimuli, is a complex lesion with three concurrences of tissue deterioration, circulatory disorder and exudation, and tissue proliferation. More specifically, inflammation is part of innate immunity, and as in other animals, innate immunity in humans recognizes cell surface patterns that are specific to pathogens. Phagocytes recognize cells having such a surface as non-self and attack the pathogen. When a pathogen breaks through the body's physical barrier, an inflammatory response occurs. An inflammatory response is a non-specific defensive action that creates a hostile environment against microorganisms invading an wound. In an inflammatory response, when a wound is formed or an external infectious agent enters the body, leukocytes in charge of an early-stage immune response gather together and express cytokines. Therefore, the expression level of intracellular cytokines is an indicator of the activation of an inflammatory response.

In one Example of the present invention, an anti-inflammatory activity of the present invention may be caused by microvesicles, exosomes, or cell secretomes included in a composition of the present invention, and more specifically, may be caused by exosomes but is not limited thereto.

As used in the present specification, the term “inflammatory disease” may be one selected from the group consisting of dermatitis, allergy, atopy, asthma, conjunctivitis, periodontitis, rhinitis, otitis media, pharyngolaryngitis, tonsillitis, pneumonia, gastric ulcer, gastritis, Crohn's disease, colitis, gout, ankylosing spondylitis, rheumatic fever, lupus, fibromyalgia, psoriatic arthritis, osteoarthritis, rheumatoid arthritis, periarthritis, tendonitis, tenosynovitis, peritendinitis, myositis, hepatitis, cystitis, nephritis, Sjogren's syndrome, multiple sclerosis, and acute or chronic inflammatory diseases, but is not limited thereto.

As used in the present specification, the term “prevention” refers to any action that suppresses symptoms or delays the onset of an inflammatory disease by administration of a composition, and “treatment” or “amelioration” refers to any action that improves symptoms of an inflammatory disease or that makes beneficial changes by administration of a composition.

Furthermore, as used in the present specification, the term “quasi-drug” refers to articles corresponding to one of textiles, rubber products, or similar products used for the purpose of treating, alleviating, caring, or preventing human or animal diseases; products that have a weak action or having no direct action on the human body and that are not instruments or machines and similar products; and products that correspond to one of the agents used for sterilization and insect extermination, and similar purposes to prevent infectious diseases, wherein the term “quasi-drug” refers to those that are not instruments, machines or apparatuses among the articles that are used for treating, alleviating, caring, or preventing human or animal diseases, and those that are not instruments, machines or apparatuses among articles that are used for the purpose of giving a pharmacological effect on the structure and function of humans or animals.

On the other hand, “excipient” usually refers to a substance added for the purpose of giving a suitable hardness or shape to a drug, or giving a certain weight when the amount of the main agent is little to make a size that is easy to handle. On the contrary, in the present invention, “excipients (also referred to as additives)” known in the art impart a function to efficiently separate extracellular vesicles from impurities such as cell debris, waste products, proteins, and macroparticles. As a non-limiting example of the present invention, an excipient may at least one selected from the group consisting of sucrose, trehalose, lactose, lactulose, maltose, cellobiose, isomaltose, turanose, dextrose, glucose, galactose, fructose, talose, mannose, tagatose, psicose, erythrose, erythrulose, threose, erythritol, arabinose, xylose, lyxose, ribose, ribulose, xylulose, aldoheptose, heptulose, octulose, gentianos, umbelliferose, planteos, isorichnose, raffinose, lychnose, stachyose, verbascose, mannitol, maltitol, lactitol, maltotriose, polyethylene glycol, xylitol, sorbitol, starch degraded sugar, starch degrading sugar reduced alcohol, non-ionic surfactants (e.g., Tween-20, Triton X-100, Pluonic F-68, etc.), histidine, glycerol, and cholesterol.

In the composition of one Example of the present invention, the type of the stem cells is not limited, but may preferably be tonsil-derived stem cells but is not limited thereto. The type of the stem cells is not limited as long as there is no risk of infection by pathogens and they do not cause immunorejection, but they may preferably be tonsil-derived stem cells.

The composition of one Example of the present invention can be effectively used for preventing, suppressing, alleviating or ameliorating inflammatory diseases.

The pharmaceutical composition of the present invention may be prepared in a unit dosage form or may be prepared by incorporating into a multi-dose container through formulation using a pharmaceutically acceptable carrier and/or excipient according to a method that can be easily performed by one of ordinary skill in the art.

Pharmaceutically acceptable carriers included in the pharmaceutical composition of the present invention are those that are commonly used in formulation, and may include lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methylcellulose, methylhydroxybenzoate, propylhydroxybenzoate, talc, magnesium stearate, mineral oil, etc., but is not limited thereto.

The pharmaceutical composition of the present invention may further comprise a lubricant, a wetting agent, a sweetening agent, a flavoring agent, an emulsifying agent, a suspending agent, a preservative, and the like in addition to the components described above. Suitable pharmaceutically acceptable carriers and agents are described in detail in Remington's Pharmaceutical Sciences (19th ed., 1995).

The pharmaceutical composition of the present invention can be administered orally or parenterally, and in the case of parenteral administration, it may be administered by intravenous injection, subcutaneous injection, intramuscular injection, intraperitoneal injection, endothelial administration, topical administration, intranasal administration, intrapulmonary administration and intrarectal administration or the like. When administered orally, since proteins or peptides are digested, an oral composition must be formulated to coat an active agent or to protect from degradation in the stomach. In addition, a pharmaceutical composition may be administered by any apparatus capable of transporting an active substance to a target cell.

A suitable dosage of a pharmaceutical composition of the present invention varies depending on factors such as formulation method, administration method, the age, weight, sex, medical conditions of the patient, food, administration time, administration route, excretion rate, and reaction sensitivity. A physician of ordinary skill can readily determine and prescribe a dosage that is effective for the desired treatment or prevention. According to a preferred embodiment of the present invention, a daily dosage of a pharmaceutical composition of the present invention is 0.001 to 100 mg/kg. As used in the present specification, the term “pharmaceutically effective amount” refers to an amount that is sufficient to prevent or treat an inflammatory disease.

The pharmaceutical composition of the present invention may be administered as an individual preventive or therapeutic agent, or in combination with another therapeutic agent, and may be administered sequentially or simultaneously with a conventional therapeutic agent.

The concentration and/or content of the pharmaceutical composition of the present invention is not particularly limited as long as it comprises the anti-inflammatory active composition described above.

In addition, the present invention provides an anti-inflammatory functional cosmetic composition comprising the anti-inflammatory active composition described above.

The cosmetic composition is not particularly limited as long as it is suitable for use in preventing or ameliorating an inflammatory disease. For example, it may be formulated into solution, suspension, emulsions paste, gel, creams lotion, powder, soap, surfactant-containing cleanser, oil, powder foundation, emulsion foundation, wax foundation, sprays, and the like, but is not limited thereto. More specifically, it may be prepared in the formulation of softening lotion, nourishing lotion, nourishing cream, massage cream, essence, eye cream, cleansing cream, cleansing foam, cleansing water, pack, spray or powder.

When the formulation of the present invention is paste, cream or gel, animal oil, vegetable oil, wax, paraffin, starch, tragacanth, cellulose derivative, polyethylene glycol, silicone, bentonite, silica, talc or zinc oxide may be used as a carrier component. When the formulation of the present invention is powder or spray, lactose, talc, silica, aluminum hydroxide, calcium silicate or polyamide powder may be used as a carrier component, and in particular, in the case of spray, chlorofluorohydrocarbon, propane/butane or dimethyl ether may be included a propellant. When the formulation of the present invention is solution or emulsion, solvent, solubilizing agent or emulsifying agent is used as a carrier component, for example, water, ethanol, isopropanol, ethyl carbonate, ethyl acetate, benzyl alcohol, benzyl benzoate, propylene glycol, 1,3-butyl glycol oil, fatty acid esters of glycerol, polyethylene glycol or fatty acid esters sorbitan. When the formulation of the present invention is suspension, a liquid diluent such as water, ethanol, and propylene glycol, a suspending agent such as ethoxylated isostearyl alcohol, polyoxyethylene sorbitol ester, and polyoxyethylene sorbitan ester, microcrystalline cellulose, aluminum metahydroxide, bentonite, agar or tragacanth, and the like may be used as a carrier component. When the formulation of the present invention is a surfactant-containing cleanser, aliphatic alcohol sulfate, aliphatic alcohol ether sulfate, sulfosuccinic acid monoester, isethionate, imidazolinium derivative, methyl taurate, sarcosinate, fatty acid amide ether sulfate, alkylamido betaine, fatty alcohol, fatty acid glyceride, fatty acid diethanolamide, vegetable oil, lanolin derivative, or ethoxylated glycerol fatty acid ester may be used as a carrier component. In addition to active ingredients and carrier components, components included in the cosmetic composition of the present invention comprise components that are commonly used in cosmetic compositions for example conventional adjuvants such as antioxidant, stabilizer, solubilizer, vitamin, pigment, and fragrance.

In addition, the present invention provides a health functional food composition for preventing or ameliorating an inflammatory disease, the health functional food composition comprising the anti-inflammatory active composition described above.

The type of the health functional food is not particularly limited as long as it is commonly manufactured and/or sold. For example, the health functional food may be meat, sausage, bread, chocolate, sweets, snacks, confectionery, pizza, ramen, other noodles, chewing gum, dairy products including ice cream, various soups, beverages, tea, drinks, alcoholic beverages, and vitamin complexes, and may be used in the form of pill, powder, granule, infusion, tablet, capsule or beverage, including all types of health functional foods in the conventional sense.

A health beverage composition of the present invention is not particularly limited in liquid components other than containing the anti-inflammatory active composition, and like conventional beverages, it may contain various flavoring agents or natural carbohydrates as additional components.

Typically, the amount of the anti-inflammatory active composition included in the health functional food composition may be 0.1 to 50% by weight, preferably 1 to 40% by weight of the total food weight. In addition, in the case of long-term ingestion for the purpose of health and hygiene or health control, it may be below the range described above, and since there is not a problem eat all in terms of safety, the active ingredient may be used in an amount over the range described above.

In addition, the present invention provides a quasi-drug composition for preventing or ameliorating an inflammatory disease, the quasi-drug composition comprising the anti-inflammatory active composition described above.

The anti-inflammatory active composition of the present invention may be added to a quasi-drug composition for the purpose of preventing or ameliorating an inflammatory disease. When the anti-inflammatory active composition of the present invention is used as an additive to a quasi-drug, the anti-inflammatory active composition may be added as it is or used together with another quasi-drug or quasi-drug component, and may be appropriately used according to a conventional method. The mixing amount of an active ingredient may be appropriately determined depending on the purpose of use (prevention, health or therapeutic treatment). Generally, the quasi-drug composition is not particularly limited as long as it is for preventing or ameliorating an inflammatory disease. For example, the quasi-drug composition may be used in manufacturing disinfectant cleaner, shower foam, mouthwash, wet wipe, detergent soap, hand wash, humidifier filler, mask, ointment, coating agent or filter filler.

The present invention is described in more detail in the Examples described below. However, the Examples described below are merely illustrative of the contents of the present invention, and are not intended to restrict or limit the scope of the present invention. What can be easily inferred by one of ordinary skill in the art from the detailed description and Examples of the present invention is interpreted as belonging to the scope of the present invention. References cited in the present invention are incorporated in the present invention as references.

### Example 1. Culture of Cells

First, the tonsil tissue was kept in a 50 ml tube containing 15 to 30 ml of 70% ethanol for 1 minute and, after removing the ethanol, washed twice with 30 ml of 1×PBS to remove the residual ethanol. After removing the PBS, the tonsil tissue was transferred to a petri dish and 5 ml of PBS was poured over the tonsil tissue.

The tonsil tissue was crushed by using tweezers and the solution containing T cells and B cells was removed. After newly adding 5 ml of PBS, the tonsil tissue was further crushed by using tweezers to remove a considerable portion of lymphocytes from the tissue through cell release. After removing most of the solution, when only connective tissue remained in the petri dish, the tissue was cut into small pieces of a size of less than 5 mm by using surgical scissors. The cut tissue was transferred to a 50 ml tube to which collagenase and DNase I were added to a total volume of 10 to 20 ml, and then the resulting mixture was incubated for 30 to 60 minutes at 37° C. in a shaking incubator. Then, a cell strainer was attached to a new 50 ml tube, and the cell suspension containing the tissue was slowly transferred from the 50 ml tube containing the tissue. PBS (20 ml) was additionally poured onto the tissue remaining in the strainer to recover the remaining cells.

The cells recovered as described above were centrifuged at 300×g for 5 minutes to remove the suspension, and the precipitated cells were suspended in 5 ml of a culture medium, and then a Percoll concentration gradient was used to recover the cells.

First, the cells were carefully suspended in the upper layer of 15% Percoll and centrifuged at 500 to 900×g for 10 to 30 minutes, and then the low-density cells of the layer located between 15% and 30% were recovered, put into a new tube, and washed with PBS once. Thereafter, centrifugation was performed at 300×g for 5 minutes and the precipitated cells were added to the culture medium to prepare cell suspension. Then, the cells were counted with Hemocytomer and suspended at a concentration of 106/ml. After that, the cells were cultured in a volume of 15 ml in a 75 cm² flask or 10 ml in a 100 mm dish. The culture medium was replaced every 3 days and subculture was performed when the fibroblast-type cells reached a confluency of 70% or higher. The subculture was performed at a number of 1,000 to 5000/cm² depending on the surface area of the culture vessel.

### Example 2. Isolation of Extracellular Vesicles from Tonsillar Stromal Cell Culture Solution

The tonsillar stromal cells obtained in Example 1 were cultured in a culture medium containing fetal bovine serum, and then, when the confluency of the culture vessel was 80% or higher, the existing medium was removed and the cells were washed with PBS at least three times. Thereafter, the culture was started by replacing the media with a serum-free medium.

After 24 to 72 hours from the beginning of the culture (typically 48 hours), the culture medium was all recovered, and then centrifuged at 300 to 900×g for 5 to 10 minutes to remove cells and cell debris.

Thereafter, the supernatant (Conditioned Medium, CM) was concentrated by 10 to 50 times by using the Minimate TFF (Tangential Flow Filtration, Pall) System, and then centrifugated at a speed of 10,000 to 20,000×g for 1 hour to 2 hours at 4° C. by using a high-speed centrifuge (Himac CR 22N, Himac-Science, Rotor Number P32ST, Takeda Hitachinaka city, Japan).

After the centrifugation, the supernatant was transferred to a separate tube and PBS or saline solution was added to the precipitated fraction, the main component of which was microvesicles, in the same volume as the concentrate before the centrifugation. Then, the cells were suspended with a pipette and preserved in a freezer until a further experiment was performed.

A separately recovered supernatant was centrifuged again at 100,000 to 200,000×g for 1 to 2 hours at 4° C. by using an ultra-high-speed centrifuge (Himac CP 80NX, Himac-Science, Rotor Number P32ST, Takeda Hitachinaka city, Japan).

After the ultra-high-speed centrifugation, the supernatant (cell secretion fraction) was recovered and PBS or saline solution was added to the precipitate, the main component of which was exosomes, in the same volume as the concentrate before the centrifugation. Then, the cells were suspended and the individual fractions were stored in a freezer until they were used in an experiment.

### Example 3. Analysis of Characteristics of Extracellular Vesicle Fraction of

Tonsillar Stromal Cell

As in Example 2, the microvesicle fraction, exosome fraction, and cell secretome fraction, isolated and extracted from the tonsillar stromal cell culture medium, were subject to NTA by using NanoSight model NS300 (Malvern Instruments) equipped with a red laser and an SCMOS camera (Malvern Instruments, Malvern, UK). The samples were diluted with 1 ml PBS, mixed well, and then the diluted samples were injected into the laser chamber. The data analysis was performed with NTA v3.2.

As shown in FIG. 1, the results showed that in the conditioned medium, which was a step before the high-speed centrifugation and ultracentrifugation, the number of particles per ml was 1×10⁹, confirming that the medium was a mixture of particles of various sizes.

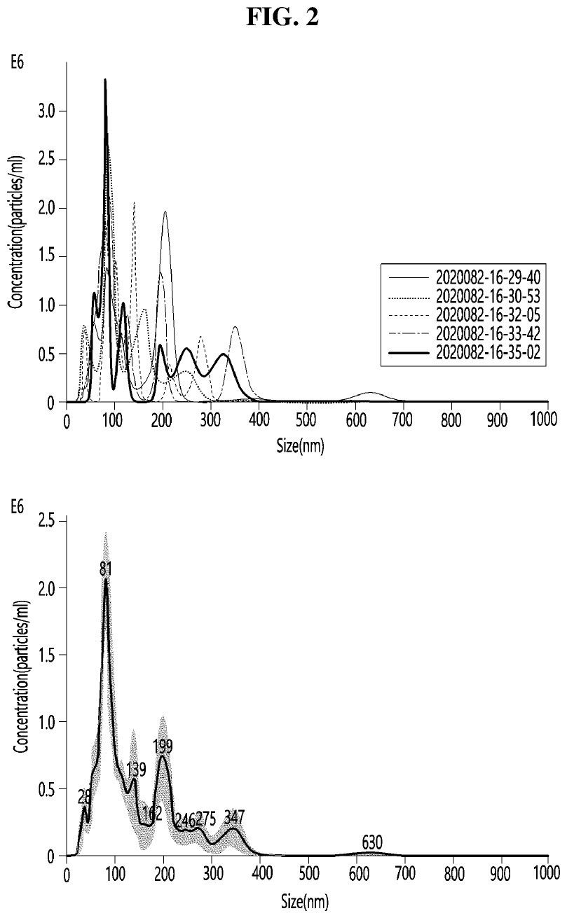

On the other hand, as shown in FIG. 2, in the microvesicle fraction, the average number of particles per ml was 1.2×10⁸ and the average particle size was 150 nm, confirming that the fraction was also a mixture of particles of various sizes.

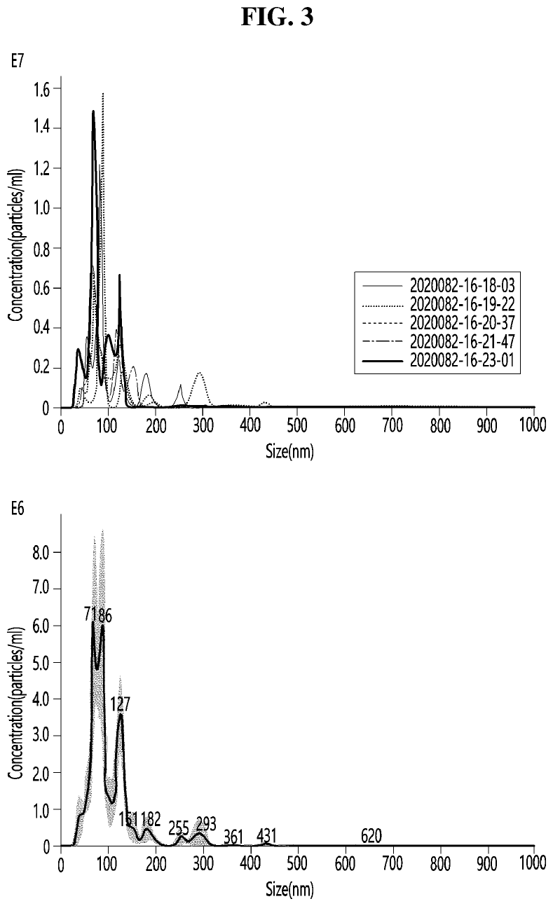

In addition, as shown in FIG. 3, it was confirmed that the exosome fraction had an average number of particles of 2.4×10⁸ particles per ml and the average particle size of the main component was 100 nm.

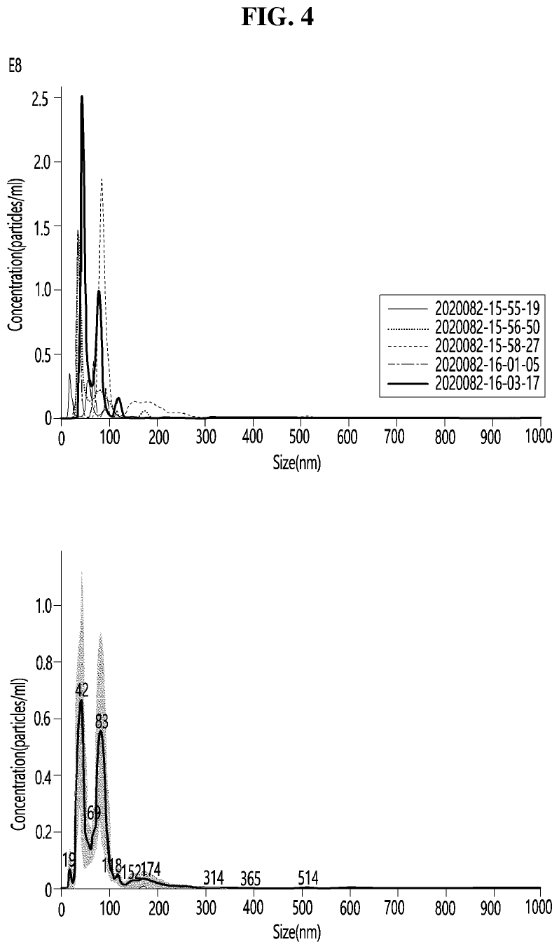

In addition, as shown in FIG. 4, in the cell secretome fraction, it was confirmed that the average particle size was 50 nm or less, which is the general size of exosomes, and 7.2×10⁹ particles per ml were contained.

### Example 4. Confirmation of Anti-Inflammatory Effect

### 4-1. RNA Extraction and Real-Time PCR (RT-qPCR)

To confirm the anti-inflammatory effect of the present invention, human peripheral blood was collected from a healthy donor, mixed with PBS at a ratio of 1:1, and overlayed on top of 20 ml of a Ficoll Hypaque (Cytiva) solution having a specific gravity of 1.077 g/ml in a 50 ml tube. Then, after 20 minutes of centrifugation at 900×g, the intermediate layer was recovered to extract peripheral blood mononuclear cells (PBMC).

After recovering the PBMC layer as described above, centrifugation was repeated twice for 5 to 10 minutes at 300×g, and the precipitated cells were suspended in RPMI-1640 (Cytiva HyClone RPMI, Logan, Utah, U.S.) supplemented with 10% FBS and 1% penicillin/streptomycin and used for an experiment.

The recovered PBMCs were prepared at a concentration of 3×10⁶ cells/ml and dispensed by 1 ml into each well of a 6-well plate. Then, a medium of 2 ml was added to the negative control group, an LPS (300 ng)-containing medium was added by 1 ml to each of the wells except the negative control group. Immediately after that, 1 ml of tonsillar stromal cell culture medium concentrate (final 5% to 20%), microvesicle concentrate (final 5% to 20%), exosome concentrate (final 5% to 20%), or cell secretome fraction (final 5% to 20%) was added to LPS-treated wells.

The monocytes stimulated by LPS were collected from the bottom surface from the incubator after 3 to 12 hours in CO₂ at 37° C. by using a cell scraper or trypsin/EDTA solution and centrifuged at 300×g for 5 minutes to recover. RNA was separated to perform RT-qPCR.

### 4-2. Confirmation of TNF-α Production Inhibitory Effect

To analyze the anti-inflammatory effect of the anti-inflammatory composition containing extracellular vesicles isolated from tonsil-derived mesenchymal stem cells as an active ingredient of the present invention, TNF-α and IL-10, known as inflammatory proteins, were set as target genes and expressed. To examine the changes, an experiment was performed as described below.

First, RNA was extracted from the cells recovered as in Example 4-1 by using BIONEER AccuPrep Universal RNA Extraction kit, and then 1 μg of RNA per condition was treated with 10 pmol of oligo-dT primer and dNTP mixture (each 10 mM) at 65° C. for 5 minutes. Then, cDNA was synthesized with a cDNA Synthesis Kit (Takara) by adding final 1× buffer, 200 UM-RTase, and 20 U RNase inhibitor in a total volume of 20 μl at 42° C. for 30 to 60 minutes. Then, the cDNA was subjected to real-time PCR in QuantStudio 3 Real-Time PCR Instrument (Thermo Fisher) with FastFire qPCR PreMix (SYBR Green, TIANGEN, Beijing, China) under the conditions of Hold Stage: 95° C. 1 min; PCR Stage: 40 cycles (step 1 at 95° C. for 5 sec and step 2 at 60° C. for 15 sec); and Melt Curve Stage: step 1 at 95° C. for 15 sec, step 2 at 60° C. for 1 min, and step 3 (dissociation) at 95° C. for 15 sec.

The expression level of TNF-α was averaged by GAPDH, and the ΔCt and ΔΔCt values were obtained. The indicated RQ value was expressed by using the 2-AAct calculation method, wherein the primers were GAPDH (FW:5′-GGAGCGAGATCCCTCCAAAAT-3′, RV: 5′-GGCTGTTGTCATACTTCTCATGG-3′), 5′-TNF-α (FW: 5′-CCCCCAGGGACCTCTCTCTAATC-3′, RV: 5′-GGRTTGCTACAACATGGGCTACA-3′).

Thereafter, each fraction of the culture medium of the stromal cells was treated in PBMC stimulated with LPS, and then the anti-inflammatory effect was analyzed by a real-time PCR.

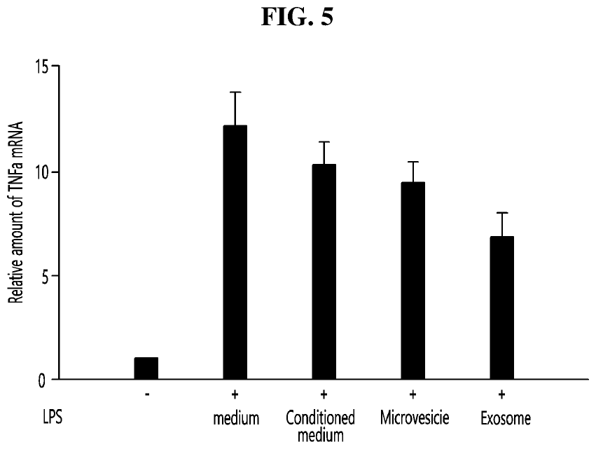

As shown in FIG. 5, the results confirmed that the anti-inflammatory composition comprising extracellular vesicles isolated from tonsil-derived mesenchymal stem cells as an active ingredient of the present invention effectively reduced the TNF-α increased by LPS. Specifically, as shown in FIG. 5, the results of the RT-PCR showed that, compared to the control group treated with only LPS, all the experimental groups treated with the conditioned medium (CM), microvesicles, and exosomes exhibited a lower level of TNF-α, and the level of TNF-α was low in the order of exosomes, microvesicles, and CM. In particular, it was confirmed that the experimental group treated with exosomes exhibited an excellent TNF-α production inhibitory effect.

### 4-3. Confirmation of IL-10 Increasing Effect

To confirm an effect of inducing the expression of IL-10, an anti-inflammatory cytokine, an experiment was conducted as described below.

First, PBMCs were treated with LPS and the stromal cell culture medium fractions. Then, to quantify the IL-10 secreted by the stimulated PBMCs and contained in the culture medium, the PBMCs were divided into 24 wells at 5×10⁵ cells/well, treated with 1× LPS 100 ng/ml and the separated stromal cell culture medium, and cultured in an incubator (5% CO₂) for 24 hours at 37° C. After 24 hours, the culture medium was fully recovered and the remaining cells were removed by centrifugation at 300×g for 5 minutes at 4° C. Thereafter, measurement was performed by using Human IL-10 Standard ABTS ELISA Development Kit (PEPROTECH, Rocky Hill, NJ, USA).

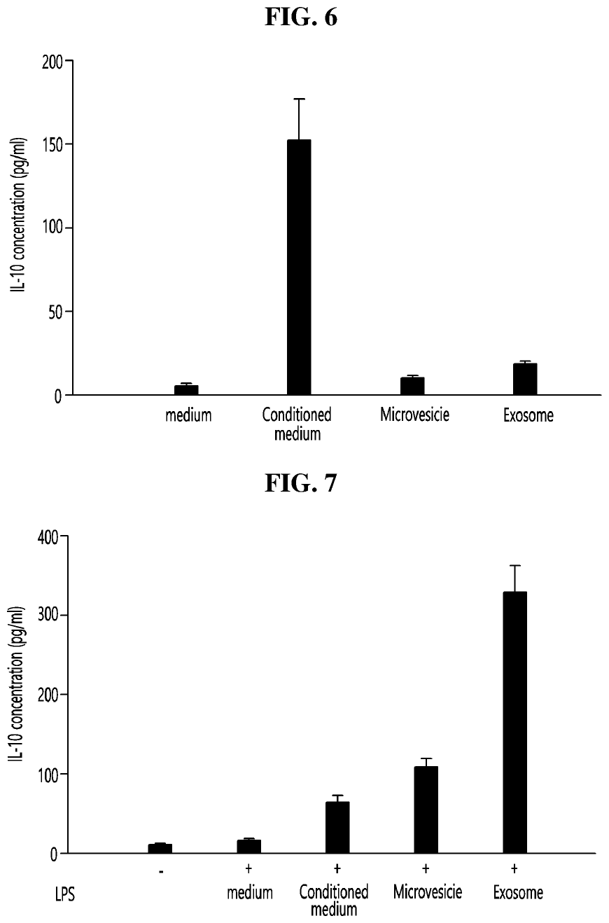

Whether the residual amount of IL-10 in the culture medium itself affects the analytical results was confirmed, and as shown in FIG. 6, the results confirmed that almost no IL-10 existed in the microvesicle fraction and the exosome fraction. These results confirmed that IL-10, which was increased by exosomes and microvesicles under the LPS-stimulation conditions, was produced by the stimulated PBMCs.

The effect of the composition of the present invention to induce the expression of IL-10, an anti-inflammatory cytokine, was tested, and the results showed that the exosome fraction had the best effect in inducing the expression of IL-10, which is known as one of the major inflammation inhibitory factors. In addition, the results showed that while the IL-10 production was very low in the PBMCs stimulated by LPS, the expression was strongly induced in the microvesicle and exosome fractions and the expression inducing effect was very high in the exosome fraction. These results indicate that the exosome fraction has the strongest inhibitory effect on skin inflammation.

Individual features of the various Examples of the present invention can be partially or entirely coupled or combined with each other, and as one of ordinary skill in the art can fully understand, various interlocking and driving operations are technically possible. The individual Examples can be implemented either independently or together with each other in an associated relationship.

Although the Examples of the present invention have been described in more detail with reference to the accompanying drawings, the present invention is not necessarily limited to these Examples, and may be variously modified and implemented without departing from the technical principle of the present invention. Therefore, the Examples described in the present invention are not intended to limit the technical principle of the present invention, but to explain the same, and the scope of the technical principle of the present invention is not limited by these Examples. Therefore, it should be understood that the Examples described above are illustrative in all respects and not restrictive. The protection scope of the present invention should be construed according to the claims below, and all technical principles within the equivalent range should be construed as being included in the scope of the present invention.

1. An anti-inflammatory active composition comprising extracellular vesicles isolated from tonsil-derived mesenchymal stem cells as an active ingredient.

2. The anti-inflammatory active composition according to claim 1, wherein the extracellular vesicles have an average diameter of 10 to 400 nm.

3. The anti-inflammatory active composition according to claim 1, wherein the extracellular vesicles have an activity of inhibiting the production of TNF-α, which is an inflammatory cytokine.

4. The anti-inflammatory active composition according to claim 1, wherein the extracellular vesicles have an activity of increasing IL-10, which is an anti-inflammatory cytokine.

5. A pharmaceutical composition for preventing or treating an inflammatory disease, the pharmaceutical composition comprising the anti-inflammatory active composition according to claim 1.

6. The pharmaceutical composition according to claim 5, wherein the inflammatory disease is one selected from the group consisting of dermatitis, allergy, atopy, asthma, conjunctivitis, periodontitis, rhinitis, otitis media, pharyngolaryngitis, tonsillitis, pneumonia, gastric ulcer, gastritis, Crohn's disease, colitis, gout, ankylosing spondylitis, rheumatic fever, lupus, fibromyalgia, psoriatic arthritis, osteoarthritis, rheumatoid arthritis, periarthritis, tendonitis, tenosynovitis, peritendinitis, myositis, hepatitis, cystitis, nephritis, Sjogren's syndrome, multiple sclerosis, and acute or chronic inflammatory diseases.

7. An anti-inflammatory functional cosmetic composition comprising the anti-inflammatory active composition according to claim 1.

8. A health functional food composition for preventing or ameliorating an inflammatory disease, the health functional food composition comprising the anti-inflammatory active composition according to claim 1.

9. A quasi-drug composition for preventing or ameliorating an inflammatory disease, the quasi-drug composition comprising the anti-inflammatory active composition according to claim 1.

10. The pharmaceutical composition according to claim 5, wherein the extracellular vesicles have an average diameter of 10 to 400 nm.

11. The pharmaceutical composition according to claim 5, wherein the extracellular vesicles have an activity of inhibiting the production of TNF-α, which is an inflammatory cytokine.

12. The pharmaceutical composition according to claim 5, wherein the extracellular vesicles have an activity of increasing IL-10, which is an anti-inflammatory cytokine.

13. The anti-inflammatory functional cosmetic composition according to claim 7, wherein the extracellular vesicles have an average diameter of 10 to 400 nm.

14. The anti-inflammatory functional cosmetic composition according to claim 7, wherein the extracellular vesicles have an activity of inhibiting the production of TNF-α, which is an inflammatory cytokine.

15. The anti-inflammatory functional cosmetic composition according to claim 7, wherein the extracellular vesicles have an activity of increasing IL-10, which is an anti-inflammatory cytokine.

16. The health functional food composition according to claim 8, wherein the extracellular vesicles have an average diameter of 10 to 400 nm.

17. The health functional food composition according to claim 8, wherein the extracellular vesicles have an activity of inhibiting the production of TNF-α, which is an inflammatory cytokine.

18. The health functional food composition according to claim 8, wherein the extracellular vesicles have an activity of increasing IL-10, which is an anti-inflammatory cytokine.

19. The quasi-drug composition according to claim 9, wherein the extracellular vesicles have an average diameter of 10 to 400 nm.

20. The quasi-drug composition according to claim 9, wherein the extracellular vesicles have an activity of inhibiting the production of TNF-α, which is an inflammatory cytokine.
